(19) United States
(12) Patent Application Publication
(10) Pub. No.: US 2022/0110579 A1
(43) Pub. Date: Apr. 14, 2022

(54) APPARATUS FOR INTRAOPERATIVE NEUROMONITORING OF NERVES IN THE PELVIC REGION OF A PATIENT

(71) Applicant: Dr. Langer Medical GMBH, Waldkirch (DE)

(72) Inventors: Andreas Langer, Waldkirch (DE); Ramona Schuler, St. Märgen (DE)

(21) Appl. No.: 17/433,368

(22) PCT Filed: Feb. 13, 2020
(86) PCT No.: PCT/IB2020/051196

§ 371 Date: Aug. 24, 2021

(30) Foreign Application Priority Data
Feb. 27, 2019 (DE) 10 2019 104 947.4

(51) Int. Cl. A61B 5/00; A61B 5/0538; A61B 5/11; A61B 5/20

(52) U.S. Cl. CPC A61B 5/4047; A61B 5/0538; A61B 5/1107; A61B 5/6873; A61B 5/202; A61B 5/4255; A61B 5/1126

(57) ABSTRACT

An apparatus for intraoperative neuromonitoring of nerves in the pelvic region of a patient, having at least one nerve stimulator which can be introduced into the operating area during an operation, and a sensor device which can be attached to at least one pelvic organ of the patient. In order to provide an apparatus of this type, by means of which it is possible to reliably monitor, without a significant delay, the integrity/proper functioning of nerves which act on one or more pelvic organs, i.e. in particular the bladder and/or the rectum, during an operation in the pelvic region of a patient, the sensor device includes at least two sensing electrodes, which can be applied to the pelvic organs, and an impedance measuring device, which is or can be connected to the sensing electrodes and has a display showing impedance changes in the pelvic organs between the sensing electrodes. The stimulation of nerves which are connected to a pelvic organ such as the bladder or the rectum leads to changes in the complex electrical resistance (impedance) in the organ tissues in question, which can act as an indicator of the integrity of a nerve stimulated by the nerve stimulator.

### BACKGROUND OF THE INVENTION

This Application claims priority in PCT Application Ser. No. PCT/I132020/051196 filed on Feb. 13, 2020, which claims priority in German patent application Ser. No. 10 2019 104 947.4 filed on Feb. 27, 2019, which are both incorporated by reference herein.

The invention relates to an apparatus for intraoperative neuromonitoring of nerves in the pelvic region of a patient, with at least one nerve stimulator insertable into the surgical area during an operation and at least one sensor device which is connectable to a pelvic organ of the patient.

In the area of the human pelvis runs a very filigree nerve plexus containing motor, sensory and autonomic nerve fibers. The risk of damage to this nerve plexus is very high during pelvic surgery (e.g., on the rectum, urinary bladder, prostate or uterus), with severely limiting consequences for the quality of life of the patients affected.

The consequences of pelvic nerve damage are postoperative functional disorders of the pelvic organs. Up to 38% of patients suffer from urinary incontinence after pelvic surgery, about 46% of patients from fecal incontinence, and 18-76% of patients from sexual dysfunction. These significant limitations in quality of life affect a significant number of patients, given the high number of surgeries performed each year (approximately 437200 surgeries on the rectum, urinary bladder, urethra, and internal genital organs in 2015, according to the Federal Statistical Office).

The pelvic nerve plexus is highly complex, its expression is extremely fine and the location of the many different nerves varies interindividually. The established methods of neuromonitoring, such as those known from thyroid surgery, cannot be readily transferred to application in the pelvic region. Particularly in rectum surgery, efforts have been made for several years to apply nerve-sparing surgical techniques, but no system for standardized and routine intraoperative nerve monitoring in the pelvic region has yet been established across the board. For the surgeon, differentiating the nerves from surrounding tissue and thus sparing the pelvic nerves is very difficult and requires a sound knowledge of neuroanatomy and neurophysiology. The different characteristics of enveloping fascia and fatty tissue in the pelvic cavity from patient to patient, as well as possible tumor infiltration, additionally prevent the identification of the pelvic nerves.

From EP 2 589 410 B1 a generic apparatus for substantially simultaneous control of the stimulability of functions controlled by the autonomic nervous system in the pelvic region is known, which has at least one stimulation electrode for intraoperative insertion into the pelvic region during an operation and in which the sensor device has as sensors at least one for measuring the bladder pressure and at least one for measuring the activity of the sphincter ani internus, the sensor for measuring the activity of the sphincter being one for EMG measurement.

The use of a pressure sensor to detect a response signal when the bladder is energized by the stimulating electrode has been shown to be disadvantageous because this method requires at least partial filling of the bladder with fluid, which is inherently undesirable during surgery. The use of a pressure sensor on/in the bladder thus generally prolongs the operation time to a not inconsiderable extent, because a quantity of fluid (Ringer's solution or the like) must first be introduced into the bladder via a bladder catheter placed before the operation before the integrity of the nerve plexus can be checked, and then drained again after the nerve(s) have been checked. Intraoperative EMG measurement of the anal sphincter has also not yet been able to establish itself in clinical routine, because the measured values obtained in this way have proven to be too unspecific, so that the desired reliability of the method is lacking. The main reason for this is that there is a large proportion of autonomous fibers in the nerve plexus in the pelvic region, the target muscle of which is a smooth muscle. Smooth muscle contains pacemaker cells (Cajal cells) that control smooth muscle activity and are modulated by autonomic fibers. Thus, smooth muscle contraction is difficult to detect by conventional EMG.

### SUMMARY OF THE INVENTION

It is the task of the invention to provide an apparatus of the type mentioned at the outset, with which it is possible to reliably and without appreciable delay check the integrity/functionality of nerves acting on one or more pelvic organ(s) , i.e. in particular the bladder and/or the rectum, during an operation in the pelvic region of a patient.

This task is solved by the invention in that the sensor device has at least two measurement electrodes which are appliable to the pelvic organ, and an impedance measurement device, which is connected or connectable to the measurement electrodes, with a display indicating impedance changes at the pelvic organ between the measurement electrodes. In this context, the phrase “appliable to the pelvic organ” is intended to cover not only direct application, but also indirect application, i.e. application to a body part connected to the pelvic organ, such as the urethra forming the outlet from the urinary bladder.

Surprisingly, it has been shown that stimulation of nerves associated with a pelvic organ, such as the bladder or rectum, results in changes in the complex electrical resistance (impedance) at the organ tissue concerned, which can serve as an indicator of the integrity of a nerve stimulated by means of the nerve stimulator. With the aid of the invention, it is possible to measure the changes in impedance that occur and to display them to the surgeon on the display, thereby providing feedback as to whether the stimulated nerve is still fully functional, i.e. intact, or whether it may have been injured during the operation.

The nerve stimulator is a preferably bipolar stimulation electrode energizable with a stimulation current, with which it is easily possible to subject the autonomic and somatic pelvic nerves to direct electrical stimulation. This provides a simple and reliable means of inducing reproducible stimulation-induced contractions on the pelvic organs. Contraction of the smooth muscles of the urinary bladder and rectum can be measured by measuring the change in tissue impedance with the invention. It is particularly advantageous in the method which can be carried out with the invention that the measurement of the change in impedance at the urinary bladder is also possible when the bladder is empty, i.e. does not contain any fluid. The invention thus enables intraoperative localization and functional control of the pelvic nerve plexus connected to the pelvic organs being monitored, without the need to first prepare them in a time-consuming manner—for example, by filling them with Ringer's solution or the like—for carrying out a check stimulation.

In an advantageous embodiment of the invention, the sensor device can comprise a test current supply device with at least two test current electrodes which are appliable to the pelvic organ. In this case, the arrangement can expediently be made, for example, in such a way that one test current electrode and one measurement electrode are each combined in one electrode unit. With such an arrangement, the measurement technique for impedance measurement can be implemented particularly advantageously using the 4 electrode technique. Preferably, a known constant current is introduced as the test current via the two test current electrodes into the body at the pelvic organ concerned. The voltage across the measurement section resulting from the tissue impedance is tapped via the two measurement electrodes. Changes in tissue impedance due to stimulation of a nerve associated with the organ result in a change in the voltage tapped from the measurement electrodes, which is visible on the display device.

The introduced constant current (hereinafter referred to as test current) for measurement on the pelvic organ (bladder, rectum) should not have any direct current components, since electrolysis may occur between the electrodes due to the fluids in the tissue when direct current is applied. The introduced constant test current is therefore preferably modulated with a specific frequency, for example a frequency of 50 kHz. For this purpose, in an advantageous further development of the invention, the test current supply device provides an alternating test current applied to the test current electrodes with a fixed or adjustable test current frequency.

In order to be able to monitor impedance changes at different organs during an operation, the test current supply device can comprise several, in particular two, test current electrode pairs, each with two test current electrodes, and means for generating test alternating currents with different test current frequencies at the different test current electrode pairs. The different test current electrode pairs can then be applied to different organs, for example on the one hand the urinary bladder and on the other hand the rectum of the patient to be operated on, in each case at suitable positions, in order to apply a first test current with a first test current frequency to one organ and a second test current with a second frequency differing from the first test current frequency to the other organ. In this way, the voltages tapped from the organs by means of measurement electrodes can be clearly associated to the organ concerned.

The sensor device preferably has a measurement signal amplifier, preferably a lock-in amplifier, particularly preferably a dual-phase lock-in amplifier, processing the measurement signal tappable by means of the measurement electrodes.

It has proved to be particularly advantageous if the impedance measurement device substantially consists of an impedance spectroscope. Impedances consist of a real part and an imaginary part, where the real part corresponds to a pure ohmic resistance. The imaginary part is determined by capacitive and inductive components and is frequency dependent. Contraction of the smooth muscle of the monitored pelvic organs may cause not only changes in the ohmic resistance between the measurement electrodes, but also changes in the capacitive component of the impedance, which can be detected particularly clearly by using an impedance spectroscope. In impedance spectroscopy, the modulation frequency of the test current is varied over a specific, defined frequency spectrum. The tissue impedance can be determined as a function of the modulation frequency. Impedance changes can thus be determined very reliably and clearly.

For particularly simple and reliable application of the electrodes to the pelvic organ to be monitored, it has proved advantageous if at least one electrode unit comprising a measurement electrode and a test current electrode is designed in the form of an electrode catheter and/or a rectal probe. An electrode catheter of this design can be inserted through the urethra in such a way that the two electrodes provided on it remain in reliable direct contact with the bladder sphincter for the duration of the operation. Similarly, the electrodes of a rectal probe designed to monitor the rectum attach to the anal sphincter when the probe has been inserted into the patient as intended. The other measurement and test current electrodes can be needle electrodes that can be applied intramurally and can be arranged, for example, as a pair of needles on a common needle electrode holder. With such a holder, they can then be inserted by the surgeon into the respective pelvic organ at a suitable location remote relative to the pair of electrodes on the catheter or rectal probe.

As already indicated above, the sensor device preferably has at least two measurement electrodes and two test electrodes each for application to the bladder and rectum of the patient.

These and other objects, aspects, features and advantages of the invention will become apparent to those skilled in the art upon a reading of the Detailed Description of the invention set forth below taken together with the drawings which will be described in the next section.

### BRIEF DESCRIPTION OF THE DRAWINGS

The invention may take physical form in certain parts and arrangement of parts, a preferred embodiment of which will be described in detail and illustrated in the accompanying drawings which forms a part hereof and wherein:

FIG. 1 a schematic overview of an apparatus according to the invention; and

FIG. 2 an exemplary graphical representation of the response signal obtained to stimulation of a pelvic nerve during impedance measurement on a pelvic organ.

### DESCRIPTION OF PREFERRED EMBODIMENTS

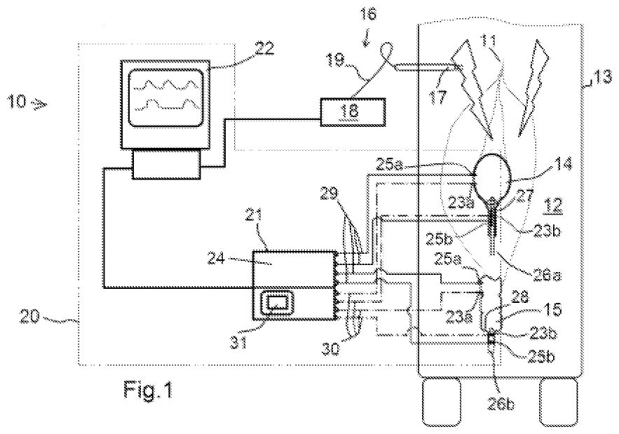

Referring now to the drawings wherein the showings are for the purpose of illustrating preferred and alternative embodiments of the invention only and not for the purpose of limiting the same, FIG. 1 schematically illustrates an apparatus, designated in its entirety as 10, for intraoperative neuromonitoring of nerves in the pelvic region of a patient. The apparatus 10 is used to localize and/or monitor nerves 11 located in the pelvic region 12 of a patient 13 and acting on one of the pelvic organs, namely in particular the urinary bladder 14 and the rectum 15, during an operation, i.e. to check them for integrity and/or functionality.

For performing intraoperative neuromonitoring, the apparatus 10 has a nerve stimulator 16 in the form of a stimulation current generator 18 and a bipolar stimulation electrode 17 hand-held by the surgeon. A stimulation current can be applied to the stimulation electrode 17, which is supplied to it by the stimulation current generator 18 via a lead 19. The apparatus 10 further comprises a sensor device, designated in its entirety as 20, having as main components an impedance measurement device 21 with a display 22 and a plurality of measurement electrodes 23 connected or connectable to the two pelvic organs urinary bladder 14 and rectum 15. The sensor device 20 further comprises a test current supply device 24 with test current electrodes 25 appliable to the two pelvic organs urinary bladder 14 and rectum 15.

In the preferred embodiment example shown, the arrangement according to the invention is such that two test current electrodes 25a, 25b and two measurement electrodes 23a, 23bare provided for each of the two pelvic organs. The arrangement is such that one test current electrode 25a (or 25b) and one measurement electrode 23a (or 23b) are combined in one electrode unit. One of the total of four electrode units is designed in the form of an electrode catheter 26a which can be inserted into the patient's urethra 27 for application to the urinary bladder 14, so that the electrodes located on the outer circumference of the catheter are applied to the inside of the urethra and thus make electrical contact with the urinary bladder. Another of the electrode units is designed as a rectal probe 26b, which is inserted into the patient's anus 28 accordingly. In the other two electrode units, the test current and measurement electrodes are designed as needle electrodes, each of which can be applied intramurally to the two pelvic organs 14, 15 at a distance from the electrode catheter 26a and the rectal probe 26b, respectively.

The test current electrodes 25 are connected to the test current supply device 24 via test current lines 29. In a corresponding manner, there are connecting lines 30 from the measurement electrodes 23 to the impedance measurement device 22, with which an integrated measurement signal amplifier 31 in the form of a conventional dual-phase lock-in amplifier is associated, with which the signals tapped from the measurement electrodes 23 are amplified and processed for a meaningful display on the screen 22. The manner in which measurement signals can be processed by means of a lock-in amplifier is known to the person skilled in the art and will not be discussed in further detail here.

In the preferred embodiment of the invention, the impedance measurement device is an impedance spectroscope with which changes in the alternating current resistance (impedance) in the pelvic organs 14, 15 between the test current electrodes 25 and the measurement electrodes 23 can be determined and visualized on the display 22 to the operator, which changes occur as a result of stimulation of the monitored nerve(s) 11 by means of the stimulation electrode 17 and a subsequent contraction of the muscle tissue of the pelvic organ concerned.

For this purpose, the procedure is as follows:

A known and constant test current (alternating current) is introduced into the smooth muscle tissue of the urinary bladder or rectum by means of the test current supply device 24 via the two test current electrodes 25a, 25b respectively applied to the pelvic organ 14 or 15. The voltage across the measurement section resulting from the tissue impedance is tapped via the two measurement electrodes 23a, 23b. Here, the introduced test current should not have any direct current components, since electrolysis can occur between the electrodes when direct current is applied due to the fluids in the tissue.

The introduced constant test current can preferably be modulated with a specific frequency (50 kHz). Since the tissue impedance is not purely ohmic but complex, a phase shift occurs between the test current and the tapped voltage across the measurement section due to the imaginary impedance component (capacitive and inductive components) in the tissue. The impedance is therefore preferably determined with the formation of the effective value/the root mean square (Root Mean Square formation).

The real part contained in the impedance of the organ tissue over the measurement section determined in the manner described corresponds to a purely ohmic resistance, while the imaginary part is determined by the capacitive and inductive components and is frequency-dependent. Changes in impedance can be clearly detected with the aid of impedance spectroscopy, for example. In impedance spectroscopy, the modulation frequency of the test current is varied over a specific, defined frequency spectrum. The tissue impedance can be determined as a function of the modulation frequency. Impedance changes can thus be detected particularly reliably.

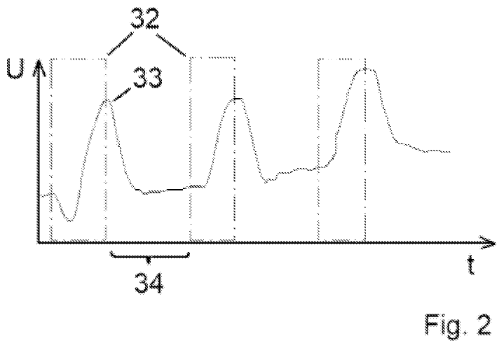

The result of an impedance measurement with the apparatus according to the invention as shown in FIG. 1 with the aid of four electrodes on each of the two pelvic organs, in response to several stimulation current pulse phases introduced at intervals into a nerve 11 to be monitored by means of the stimulation electrode 17, is shown graphically in FIG. 2. As a result of a stimulation current pulse phase 32, which is expediently a sequence of square-wave pulses with a fixed pulse duration of a pulse of, for example, 1 ms and a defined frequency of the sequence of, e. g., 30 Hz, a transmission of the signal to the smooth muscle tissue of the affected pelvic organ 14 and/or 15 occurs at the nerve 11 monitored for integrity/function, with the consequence of a stimulation-induced muscle contraction and an associated change in the impedance over the measurement distance between the measurement electrodes 23a, 23bapplied to the organ. Accordingly, the voltage tapped between the measurement electrodes changes with a short, temporal delay with respect to the time of the stimulation current pulse phase, which is displayed to the operator on the display 22, for example in a display according to FIG. 2, in which the change in impedance or the voltage increase indicating this is clearly recognizable as a deflection 33 in the voltage curve. The renewed stimulation of the nerves 11 is already possible while maintaining a rest phase 34 of less than 120 seconds, during which the contraction of the smooth muscle subsides and thus again the impedance increases over the measurement distance.

With the aid of the apparatus according to the invention, it is thus possible in a reliable manner to check nerves in the pelvic region of a patient, in particular in the lesser pelvis, for their integrity or functional capability, because an absence of a change in impedance at the pelvic organ in question despite a triggered stimulation current pulse is an indication that the nerve tracts between the stimulation site at the nerve and the pelvic organ observed are damaged or completely interrupted, for example because they have been cut with a scalpel. The surgeon can also use the apparatus to locate nerves in the surgical area, which is often quite difficult to survey, in order to decide whether a planned incision can be made without risk of damage to the nerve.

While considerable emphasis has been placed on the preferred embodiments of the invention illustrated and described herein, it will be appreciated that other embodiments, and equivalences thereof, can be made and that many changes can be made in the preferred embodiments without departing from the principles of the invention. Furthermore, the embodiments described above can be combined to form yet other embodiments of the invention of this application. Accordingly, it is to be distinctly understood that the foregoing descriptive matter is to be interpreted merely as illustrative of the invention and not as a limitation.

1-10. (canceled)

11. An apparatus for intraoperative neuromonitoring of nerves in the pelvic region of a patient, with:
at least one nerve stimulator insertable into the surgical area during an operation, and
a sensor device connectable to at least one pelvic organ of the patient,
wherein the sensor device has at least two measurement electrodes, which are appliable to the pelvic organ, and an impedance measurement device, which is connected or connectable to the measurement electrodes, with a display indicating impedance changes at the pelvic organ between the measurement electrodes.

12. The apparatus according to claim 11, wherein the sensor device comprises a test current supply device with at least two test current electrodes which are appliable to the pelvic organ.

13. The apparatus according to claim 12, wherein one of the test current electrodes and one of the measurement electrodes are each combined in an electrode unit.

14. The apparatus according to claim 13, wherein the test current supply device provides an alternating test current applied to the test current electrodes with a fixed or adjustable test current frequency.

15. The apparatus according to claim 12, wherein the test current supply device provides an alternating test current applied to the test current electrodes with a fixed or adjustable test current frequency.

16. The apparatus according to claim 14, wherein the test current supply device comprises a plurality of, in particular two, test current electrode pairs, each with two test current electrodes, and means for generating test alternating currents with different test current frequencies at the different test current electrode pairs.

17. The apparatus according to claim 12, wherein the test current supply device comprises a plurality of, in particular two, test current electrode pairs, each with two test current electrodes, and means for generating test alternating currents with different test current frequencies at the different test current electrode pairs.

18. The apparatus according to claim 11, wherein the sensor device has a measurement signal amplifier processing the measurement signal tappable at the measurement electrodes.

19. The apparatus according to claim 11, wherein the sensor device has a lock-in amplifier processing the measurement signal tappable at the measurement electrodes.

20. The apparatus according to claim 19, wherein the lock-in amplifier is a dual-phase lock-in amplifier.

21. The apparatus according to claim 11, wherein the impedance measurement device substantially comprises an impedance spectroscope.

22. The apparatus according to claim 21, wherein at least one electrode unit which comprises one measurement electrode and one test current electrode each is designed in the form of an electrode catheter and/or a rectal probe.

23. The apparatus according to claim 13, wherein at least one electrode unit which comprises one measurement electrode and one test current electrode each is designed in the form of an electrode catheter and/or a rectal probe.

24. The apparatus according to claim 11, wherein the nerve stimulator is a bipolar stimulation electrode energizable with a stimulation current.

25. The apparatus according to claim 11, wherein the sensor device has at least two measurement electrodes and two test electrodes each for application to the bladder and rectum of the patient.
